(19) United States
(12) Patent Application Publication
(10) Pub. No.: US 2023/0043041 A1
(43) Pub. Date: Feb. 9, 2023

(54) SYRINGE STICTION BREAK DETECTION

(71) Applicant: Smiths Medical ASD, Inc., Plymouth, MN (US)

(72) Inventors: Sameer Pai, Plymouth, MN (US); Paul Harrison Coons, Sunfish Lake, MN (US)

(21) Appl. No.: 17/790,688

(22) PCT Filed: Dec. 22, 2020
(86) PCT No.: PCT/US2020/070948

§ 371 Date: Jul. 1, 2022

Related U.S. Application Data
(60) Provisional application No. 62/956,685, filed on Jan. 3, 2020.

(51) Int. Cl. A61M 5/142; A61M 5/168

(52) U.S. Cl. CPC A61M 5/142; A61M 5/16831

(57) ABSTRACT

An infusion system and method configured to identify a break in static friction between a plunger and an infusate cartridge during an infusion of infusate for the purpose of providing a more consistent flow of infusate and promoting a more efficient use of energy. The infusion system and method can include monitoring of force between a drive mechanism and the plunger for a decrease in a rate of the force over time during actuation of the drive mechanism, thereby indicating a break in static friction between the plunger and the cartridge, and determining a low power consumption sleep duration based on an advancement of the actuator following the break in static friction.

### CROSS-REFERENCE TO RELATED APPLICATIONS

The present application claims priority to U.S. Provisional Application No. 62/956,685, filed on Jan. 3, 2020, which is hereby fully incorporated herein by reference.

### TECHNICAL FIELD

The present disclosure relates generally to infusion pump systems, and more particularly to systems and methods for detecting when a plunger-cartridge interface breaks static friction and the plunger begins to move within the cartridge.

### BACKGROUND

Various types of infusion pumps have been useful for managing the delivery and dispensation of a prescribed amount or dose of a drug, fluid, fluid like substance, or infusate (herein, collectively, an “infusate”) to patients. Infusion pumps provide significant advantages over manual administration by accurately delivering infusates over an extended period of time. Infusion pumps are particularly useful for treating diseases and disorders that require regular pharmacological intervention, including cancer, diabetes, and vascular, neurological, and metabolic disorders. Infusion pumps also enhance the ability of healthcare providers to deliver anesthesia and manage pain. Infusion pumps are used in various settings, including hospitals, nursing homes, and other short-term and long-term medical facilities, as well as in residential care settings. There are many types of infusion pumps, including ambulatory, large-volume, patient controlled anesthesia (PCA), elastomeric, syringe, enteral, and insulin pumps. Infusion pumps can be used to administer medication through a variety of delivery methods, including intravenously, intraperitoneally, inter-arterially, intradermally, subcutaneously, in close proximity to nerves, and into an inter-operative site, epidural space, or subarachnoid space.

One type of pump that has been developed is a micro-infusion pump. Micro-infusion pumps are small, typically ambulatory pumps, that may be carried under a patient's clothing or otherwise very near to an injection site on the patient. Micro-infusion pumps are capable of reliably delivering low infusate flow rates and are often used for multiple consecutive days. In some cases, the pumps utilize replaceable cartridges, into which a plunger advances to administer the infusate.

In order to maintain a long battery life, micro-infusion pump systems typically deliver infusate in short bursts, with long, low power consumption pauses between the bursts. However, because the short bursts represent very small advances of the plunger within the cartridge (e.g., 3 μm or less), it is possible that the force applied to the plunger can cause the plunger to temporarily deform or strain in the direction of the applied force, rather than overcoming the static friction between the plunger and the cartridge to move the plunger forward. In some cases, this strain may build up over multiple consecutive short bursts until the static friction between the plunger and cartridge is overcome. Failure of the applied force to overcome the static friction within the plunger-cartridge interface can result in large intervals between infusate delivery to the patient, which can negatively affect therapeutic outcomes.

The present disclosure addresses this concern.

### SUMMARY OF THE DISCLOSURE

Embodiments of the present disclosure provide infusion pump systems and methods configured to detect when a plunger-cartridge interface breaks static friction and the plunger begins to move within the cartridge for the purpose of minimizing delays between infusate administrations, while optimizing system energy usage. For example, in one embodiment, the system and method can apply a delivery force until the static friction between the plunger and cartridge is broken and the plunger advances within the cartridge, thereby delivering a short burst of infusate. The system and method can then determine a subsequent low power consumption “sleep” duration based on how far the plunger advanced within the cartridge after the static friction force was broken.

Because the determined low power consumption sleep durations are based on an actual advancement of the plunger within the cartridge, the low power consumption sleep durations can vary in length from cycle to cycle. Further, because the advancement of the plunger within the cartridge is a positive indication of infusate delivery, the low power consumption sleep durations can be extended in length, particularly in comparison to the fixed length, low power consumption pauses common to micro-infusion pumps of the prior art. Because the low power consumption sleep durations are generally longer, fewer energy consuming wake cycles in which the plunger actively advances occur within a given period of time (e.g., a day). Minimizing the number of wake cycles reduces energy consumption, thereby enabling a longer battery life. Accordingly, embodiments of the present disclosure have the capability to both reduce the time intervals between infusate deliveries, while optimizing energy utilization.

In some embodiments, the systems and methods are further configured to enable improved occlusion detection. As referenced in the Background section, one noted problem with infusion pumps of the prior art is that breakage of the plunger-cartridge static friction force is often not predictably reliable; as a result, the force measured by an occlusion detection sensor may be a combination of both the infusate pressure and the static friction force. By contrast, because infusion pumps of the present disclosure are configured to positively determine if and when the static friction between the plunger-cartridge interface has been broken, the occlusion detection sensor can isolate measurement of the infusate pressure, thereby reducing occlusion detection “noise,” to allow for improved, shorter occlusion detection times.

An embodiment of the present disclosure provides an infusion pump configured to identify a threshold of motion of a plunger during infusate delivery from the pump. The infusion pump can include (i) an infusate vessel or cartridge including a plunger, (ii) a drive mechanism configured to actuate the plunger, (iii) a force sensor configured to monitor a force between the drive mechanism and the plunger, and (iv) a control unit. In embodiments, the infusate cartridge may be provided separately from the infusion pump. The control unit can be configured to monitor data received from the force sensor to determine a decrease in a rate of the monitored force over time during actuation of the drive mechanism, thereby indicating a break in static friction between the plunger and the infusate vessel or cartridge, and to determine a low-power consumption sleep duration based on an advancement of the actuator following the break in static friction. In one embodiment, the control unit is further configured to utilize the magnitude of the monitored force at the break in static friction to reduce noise during occlusion detection.

In an embodiment, the control unit is further configured to initiate a low-power consumption mode for the determined sleep duration. In an embodiment, the control unit is further configured to initiate actuation of the drive mechanism following the determined sleep duration. In an embodiment, the infusion pump further includes a battery. In an embodiment, a length of the sleep duration is determined to reduce the number of actuation cycles of the drive mechanism within a fixed period of time to promote a more efficient use of the battery.

In an embodiment, the control unit is further configured to apply low-pass filter to data representing the monitored force to reduce noise within the data. In an embodiment, the control unit is further configured to calculate a derivative of data representing the monitored force to determine a rate in the force over time. In an embodiment, the control unit is further configured to determine if the derivative of the data is less than a predefined threshold, thereby indicating a decrease in the rate of the force over time. In an embodiment, the predefined threshold is between about −0.025 and about −2.0.

Another embodiment of the present disclosure can provide an infusion system configured to identify a break in static friction between a plunger and an infusate vessel or cartridge during infusion of infusate for the purpose of providing a more consistent flow of infusate and promoting a more efficient use of energy. The infusion system can include (i) an infusate vessel or cartridge including a plunger, the infusate cartridge configured to retain an infusate therein, and (ii) an infusion pump. The infusion pump can be configured to selectively receive the infusate cartridge and further configured to identify a break in static friction between the plunger and the infusate cartridge during an infusion of the infusate for the purpose of providing a more consistent flow of the infusate and promoting a more efficient use of energy, the infusion pump comprising a drive mechanism configured to actuate the plunger, a force sensor configured to monitor a force between the drive mechanism and the plunger, and a control unit. The control unit can be configured to: apply a low-pass filter to data received from the force sensor to reduce noise within the data; calculate a derivative of the data to determine a rate in the force over time; determine if the derivative of the data is less than a predefined threshold, thereby indicating a break in static friction between the plunger and the infusate vessel or cartridge; and determine a low-power consumption sleep duration based on an advancement of the actuator following the break in static friction.

Yet another embodiment of the present disclosure provides a method of identifying a threshold of motion of a plunger during infusion of an infusate. The method can include: monitoring a force between a drive mechanism and a plunger for a decrease in a rate of force overtime during a steady-state actuation of the drive mechanism, thereby indicating a break in static friction between the plunger and the infusate vessel or cartridge; and determining a low-power consumption sleep duration based on an advancement of the actuator during the break in static friction.

It should be understood that the individual steps used in the methods of the present disclosure may be performed in any order and/or simultaneously, as long as the steps and methods remain operable. Furthermore, it should be understood that the apparatus and methods of the present disclosure can include any number, or all, of the described embodiments, as long as the disclosure remains operable.

The summary above is not intended to describe each illustrated embodiment or every implementation of the present disclosure. The figures and the detailed description that follow more particularly exemplify these embodiments.

### BRIEF DESCRIPTION OF THE DRAWINGS

The disclosure can be more completely understood in consideration of the following detailed description of various embodiments of the disclosure, in connection with the accompanying drawings, in which:

FIG. 1 is a perspective view depicting an infusion pump system attached to a patient, in accordance with an embodiment of the disclosure.

FIG. 2 is an exploded, perspective view depicting an infusion pump, in accordance with an embodiment of the disclosure FIG. 3 is a graphical representation depicting a force monitored by a sensor of an infusion pump system having stiction detection, in accordance with an embodiment of the disclosure.

FIG. 4 is a flowchart depicting a method of identifying a threshold of motion of a plunger during infusion of an infusate, in accordance with an embodiment of the disclosure.

FIG. 5A is a flowchart depicting a conventional infusion cycle of the prior art, without stiction detection.

FIG. 5B is a flowchart depicting an infusion cycle with stiction detection, in accordance with an embodiment of the disclosure.

FIG. 6A is a graphical representation depicting a conventional infusion cycle of the prior art, without stiction detection.

FIG. 6B is a graphical representation depicting an infusion cycle with stiction detection, in accordance with an embodiment of the disclosure.

While embodiments of the disclosure are amenable to various modifications and alternative forms, specifics thereof shown by way of example in the drawings will be described in detail. It should be understood, however, that the intention is not to limit the disclosure to the particular embodiments described. On the contrary, the intention is to cover all modifications, equivalents, and alternatives falling within the spirit and scope of the subject matter as defined by the claims.

### DETAILED DESCRIPTION

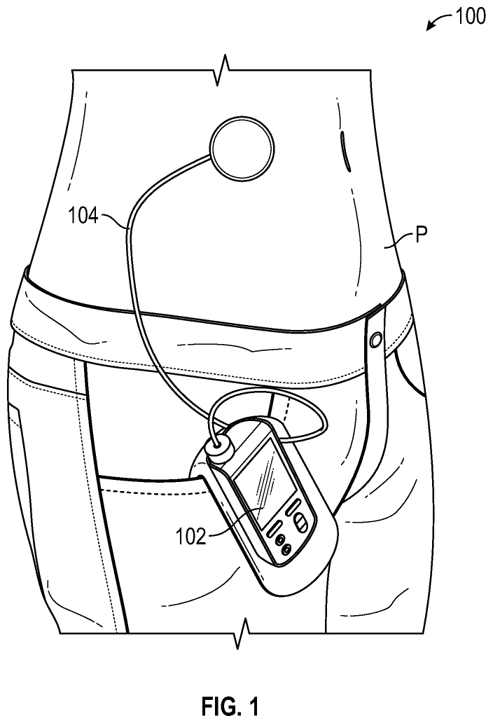

Referring to FIG. 1, an infusion pump system 100 for administering infusate to a patient (P) is depicted in accordance with an embodiment of the disclosure. The infusion pump system 100 can include an infusion pump 102 configured to control delivery of infusate to the patient P via an infusion set 104 or other tubing fluidly coupled between the pump 102 and the patient P.

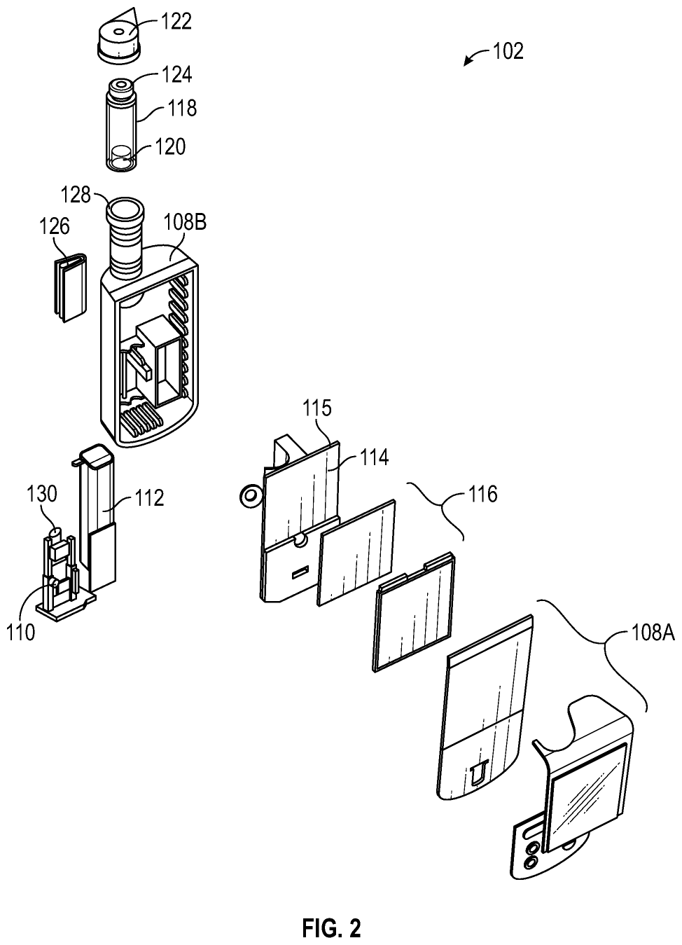

Referring to FIG. 2, an exploded, perspective view of the infusion pump 102 is depicted in accordance with an embodiment of the disclosure. In one embodiment, the infusion pump 102 can include a front housing 108A and rear housing 108B configured to provide a chassis to which other components of the infusion pump 102, such as a drive mechanism 110, power source 112, control unit 114, memory 115, and graphical user interface 116, can be assembled.

The infusion pump 102 can further include or be operably coupled to a cartridge 118 containing an infusate, which can include a plunger 120 for expulsing the infusate therefrom. The cartridge 118 can be any suitable container, vessel or other source containing or supplying a quantity of infusate. In some embodiments, the cartridge 118 can be selectively removed and replaced as needed, for example upon depletion of a supply of infusate therein. An infusion set connector 122 can fluidly couple a dispensing end 124 of the cartridge 118 to the infusion set 104.

The plunger 120 can be driven by the drive mechanism 110, for example via a lead screw arrangement configured to cooperatively actuate the plunger 120, thereby driving fluid from the cartridge 118. The drive mechanism 110 can be powered by the power source 112. In some embodiments, the power source 112 can be in the form of a battery, which can be selectively removed and replaced via a battery door 126. The drive mechanism 110 can be controlled via the control unit 114.

The control unit 114 can be any suitable programmable device that accepts digital data as input, is configured to process the input according to instructions or algorithms, and provides results as outputs. In an embodiment, the control unit 114 can be a central processing unit (CPU) configured to carry out the instructions of a computer program. In an embodiment, the control unit 114 can be an advanced RISC (Reduced Instruction Set Computing) Machine (ARM) processor or other embedded microprocessor. In an embodiment, the control unit 114 comprises a multi-processor cluster. Control unit 114 is therefore configured to perform at least basic arithmetical, logical, and input/output operations.

The memory 115 can comprise volatile or nonvolatile memory as required by the coupled control unit 114 to not only provide space to execute the instructions or algorithms, but to provide the space to store the instructions themselves. In embodiments, volatile memory can include random-access memory (RAM) dynamic random access memory (DRAM) or static random access memory (SRAM) for example. In embodiments, nonvolatile memory can include read-only memory, flash memory, ferroelectric RAM, hard disk, floppy disk, magnetic tape, or optical disk storage, for example. The foregoing examples in no way limit the type of memory that can be used, as the embodiments are given only by way of example and are not intended to limit the subject matter hereof.

The control unit 114 can receive inputs from the graphical user interface 116, which in an embodiment can be a touchscreen input and display system. In an embodiment, the control unit 114 can be in communication with an antenna 128 configured to send and receive data wirelessly to one or more external computing devices, such as a mobile computing platform (e.g., smart phone, tablet, personal computer, etc.) and/or a network. In an embodiment, the antenna 128 can be an RFID coil; although other types of antennas are also contemplated.

In an embodiment, the control unit 114 can additionally receive inputs from other input devices, sensors and monitors, such as a sensor 130. In some embodiments, the sensor 130, which can be positioned in-line with the drive mechanism 110, can be configured to monitor a force between the drive mechanism 110 and the plunger 120, and/or the position of the plunger 120 relative to the cartridge 118 according to system specifications. The sensor 130 can comprise a force sensor, pressure sensor, distance sensor, proximity sensor, or any other suitable sensor. In some embodiments, the sensor 130 can additionally function as an occlusion detection sensor configured to sense when a fluid pressure of the infusate exceeds a predefined threshold, thereby indicating the likelihood of an occlusion in the cartridge 118 and/or infusion set 104.

It should be appreciated that a more detailed explanation of the components of the infusion pump system 100, instructions regarding how to attach and use the various components of the system 100, methods for installing the related components of the system 100, and certain other items and/or techniques necessary for the implementation and/or operation of the various components of the system 100 are not provided herein because such background information is known to one of ordinary skill in the art. Therefore, it is believed that the level of description provided herein is sufficient to enable one of ordinary skill in the art to understand and practice the systems, methods and/or apparatuses as described herein.

The infusion pump 102 depicted in FIGS. 1 and 2 is an example of an ambulatory type of pump that can be used to deliver a wide range of therapies and treatments. Such ambulatory pumps can be comfortably worn by or otherwise removably coupled to a user for in-home ambulatory care by way of belts, straps, clips or other simple fastening mechanisms; and can also be alternatively provided on an ambulatory pole mounted arrangement within hospitals and other medical care facilities.

In an embodiment, the infusion pump 102 can be a micro-infusion pump configured to provide intermittent infusions of small doses of medication over an extended period of time. In a non-limiting embodiment, the infusion pump system 100 can be configured to administer treprostinil (marketed under the name Remodulin® by United Therapeutics Corporation), or other infusates for the treatment of pulmonary arterial hypertension (PAH), either subcutaneously or intravenously; although the administration of drugs other than treprostinil is also contemplated.

The embodiment of the infusion pump 102 depicted in FIGS. 1 and 2 is provided only by way of example, and is not intended to limit the scope of the subject matter hereof. Other types of pumps and other pump configurations can be utilized in various embodiments. Additionally, it should be appreciated that the systems and methods as described herein, particularly those configured to identify a break in static friction between a plunger and an infusate vessel or cartridge for the purpose of providing a more consistent flow of medication and promoting a more efficient use of energy, can equally be applied to other types of infusion pumps, particularly syringe pumps and other types of infusion pumps configured to administer infusate via an advancing plunger within a syringe, cartridge or other vessel containing infusate.

With reference to FIG. 3, a graphical representation of a force (F) monitored by the sensor 130 over time (t) is depicted in accordance with an embodiment of the disclosure. Time t in seconds is depicted along the x-axis, while the force F sensed between the drive mechanism 110 and the plunger 120 in lbs is depicted along the y-axis.

At t₀, the magnitude of the sensed force F can begin at an established baseline, which in some embodiments can represent a fluid pressure of the infusate under ambient conditions, for example where the infusate is able to freely flow through the infusion set 104 (free from occlusions) into the vasculature or other selected infusion site of a patient, until a pressure within the cartridge 118 and the selected infusion site reach an equilibrium. Accordingly, in an embodiment, the baseline force magnitude at to can represent a blood or other bodily fluid pressure at the infusion site, with the addition of a relatively small force factor to account for a flow resistance within the infusion set 104.

Actuation of the drive mechanism 110 can begin at to. In some embodiments, actuation of the drive mechanism 110 can be a constant, non-variable, steady state, and/or linear actuation. As the drive mechanism 110 actuates, it applies a force F to the plunger 120 in an attempt to translate or shift the plunger 120 within the cartridge 118. In some embodiments, however, the plunger 120 remains stationary relative to the cartridge 118 until the force F exceeds an opposing static friction force (occasionally referred to herein as “stiction”) present in the interface between the cartridge 118 and the plunger 120.

FIG. 3 depicts an increase in force F between t₀ and t₁, until (at t₁) the force F exceeds the opposing stiction. Although the force F is depicted as being substantially linear between to and t₁, other factors, such as a natural resiliency or material strain characteristics of the plunger 120 and/or drive mechanism 110 may have an impact on the measurable force F, thereby affecting the overall shape of the force curve.

At t₁, the force meets or exceeds the opposing stiction between the plunger 120 and the cartridge 118 to establish a threshold of motion. Thereafter, the plunger 120 moves relative to the cartridge 118, thereby applying a portion of the force F to the infusate, so as to urge infusate through the dispensing end 124 of the cartridge 118 and ultimately into the infusion set 104. Initially, after the opposing stiction has been overcome, the measurable force F decreases, as can be seen between t₁ and t₂. In some cases, this can be caused by the plunger 120 lurching or suddenly moving forward upon a break in the stiction (occasionally referred to herein as “stiction breakage” or “break”).

Between t₂ and t₃, as the plunger 120 advances forward, the force F approximates the running friction, which can be a combination of a reactive fluid pressure of the infusate and kinetic friction between the cartridge 118 and the plunger 120. Slow advancement of the plunger 120 may cause the plunger to momentarily stick (e.g., stiction re-engages) and then slip (e.g., stiction breaks), causing the force F to oscillate (as depicted in FIG. 3). Alternatively, faster advancement of the plunger 120 may result in a more constant force F between t₂ and t₃.

It should be appreciated that the terms “plunger-cartridge interface,” “static friction,” “stiction,” “stiction breakage,” and “break” are used for basic explanation and understanding of the operation of the systems, methods and apparatuses of this disclosure. Therefore the terms “plunger-cartridge interface,” “static friction,” “stiction,” “stiction breakage,” and “break” are not to be construed as limiting the systems, methods, and apparatuses of this disclosure, and are to be understood broadly to include any infusion pump system having an ability to sense when a sufficient amount of force has been developed within the pump to confirm that a threshold of motion has been established between a plunger and a vessel, container, or cartridge containing the plunger.

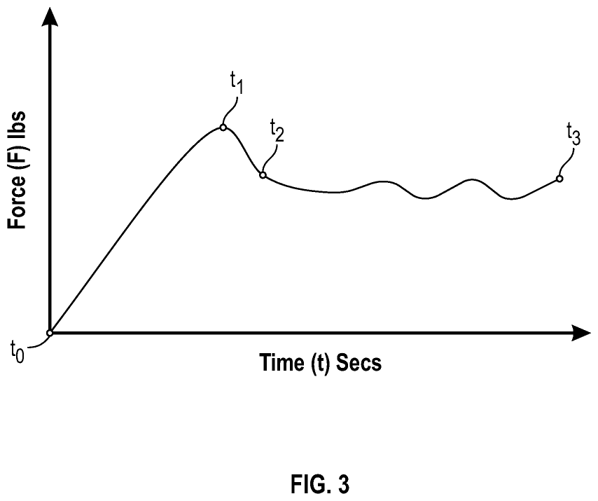

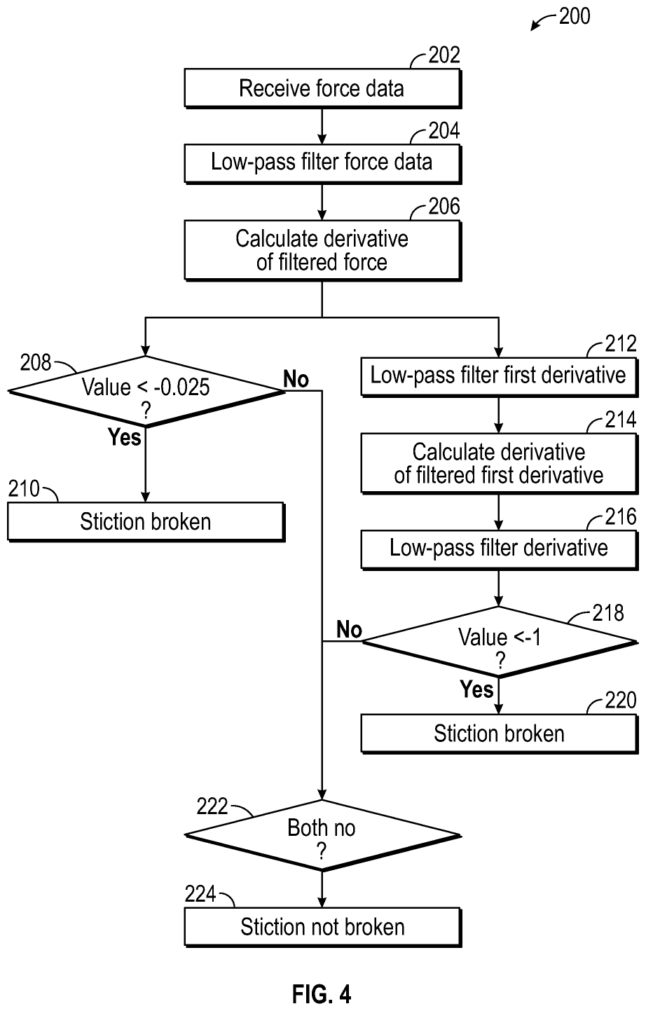

Referring to FIG. 4, a flow chart depicting a method 200 of identifying a threshold of motion of a plunger 120 during delivery of an infusate, is depicted in accordance with an embodiment of the disclosure. At 202, data from the sensor 130 can be received. In an embodiment, the data can represent a measurable force between the drive mechanism 110 and the plunger 120, which can be received by the control unit 114 and/or memory 115.

At 204, the data can be run through a low-pass filter to reduce higher frequency oscillations (sometimes referred to as “noise”) within the data. In a non-limiting example, the low pass filter can utilize a first-order Butterworth filter with a critical frequency of 0.0042 multiplied by the Nyquist frequency.

At 206, a first derivative can be calculated for the data to determine a change in rate in the force over time (δF/δt). At 208, the first derivative can be compared to a predefined threshold to determine if the first derivative of the data is less than a predefined threshold, thereby indicating stiction breakage between the plunger 120 and the cartridge 118. FIG. 4 depicts the predefined threshold as −0.025; although a range of other predefined threshold values are also contemplated. At 210, stiction breakage is verified.

At 212, the first derivative can be run through a low-pass filter to reduce higher frequency oscillations or noise within the calculated first derivative. At 214, a second derivative of the filtered first derivative can be calculated. At 216, the second derivative can be run through a subsequent low-pass filter to reduce higher frequency oscillations or noise within the calculated second derivative. At 218, the filtered second derivative can be compared to a second predefined threshold to determine if the filtered second derivative is less than the second predefined threshold, thereby indicating stiction breakage between the plunger 120 and the cartridge 118. FIG. 4 depicts the second predefined threshold as −1.0; although a range of other predefined threshold values are also contemplated. At 220, stiction breakage is verified. In some embodiments, the actions represented in 212-220 can be performed simultaneously, or in conjunction with, other acts performed during the method 200, as long as the method 200 remains operable.

At 222, the decisions made at 208 and 218 can be compared. At 224, if both the first derivative of the data is greater than the first predefined threshold, and the filtered second derivative is greater than the second predefined threshold, it can be concluded that the stiction has not been broken. In some embodiments, the method 200 operates as a continuous loop until actuation of the drive mechanism 110 ceases.

It should be understood that the individual steps used in the methods described by example or otherwise contemplated herein may be performed in any order and/or simultaneously, as long as the steps and methods remain operable. Furthermore, it should be understood that the apparatus, devices, systems, and methods of the present disclosure can include any number, or all, of the described embodiments, as long as the disclosure remains operable.

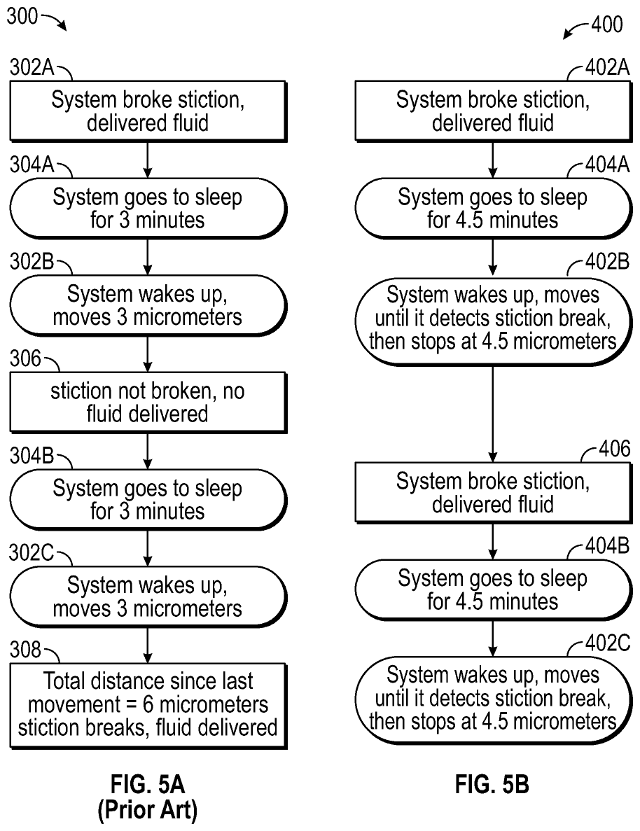

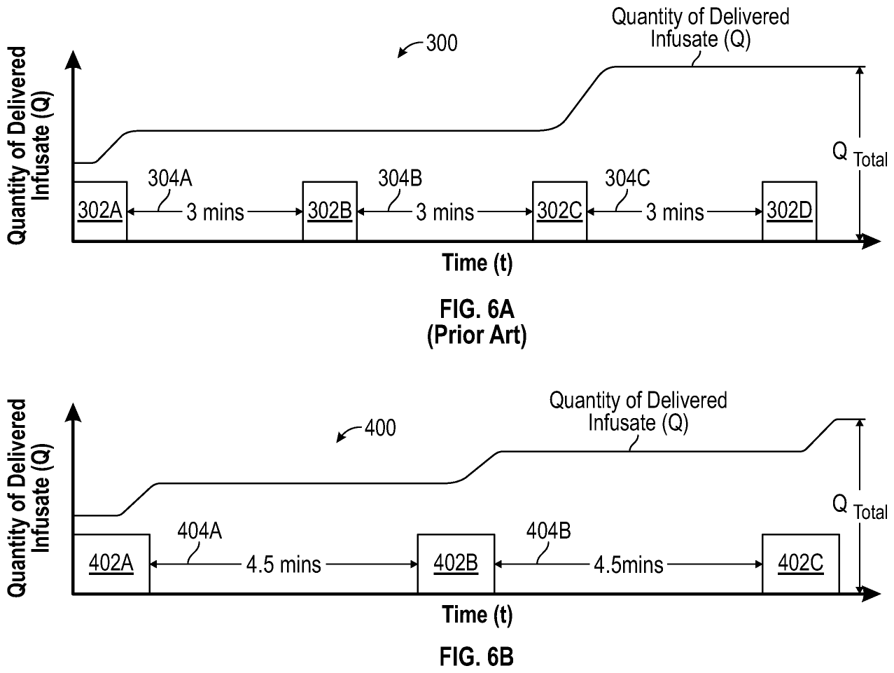

FIGS. 5A and 6A respectively depict an operational flowchart and a graphical representation of a conventional infusion cycle 300 without stiction break detection. By contrast, FIGS. 5B and 6B respectively depict an operational flowchart and a graphical representation of an infusion cycle 400 with stiction break detection, in accordance with an embodiment of the disclosure. As can be seen, the infusion cycle 400 with stiction break detection (depicted in FIGS. 5B and 6B) provides a more consistent delivery of infusate Q than the conventional infusion cycle 300 (depicted in FIGS. 5A and 6A), with longer sleep durations and fewer wake cycles within a fixed period of time, thereby representing an improvement in energy efficiency over the conventional infusion cycle 300.

With reference to FIG. 5A, a conventional infusion cycle 300 begins at 302A with a drive mechanism actuation cycle in which static friction is broken, such that a quantity of infusate is successfully delivered. At 304A, the system enters a low power consumption sleep duration for a preestablished length of time (e.g., 3 mins). At 302B, the system wakes up and begins a second actuation cycle. However, as noted at 306, static friction is not broken, so no infusate is actually delivered; in part, this is because each drive mechanism actuation cycle 302 is configured to apply the equivalent force to advance a predefined distance (e.g., 3 μm), regardless of whether any infusate is actually dispensed or delivered prior to completion of the actuation cycle 302. Accordingly, the conventional infusion cycle 300 operates “in the blind” with regard to actual movement of a plunger relative to a cartridge containing the plunger.

At 304B, the system enters a second sleep duration for the preestablished length of time (e.g., 3 mins). At 302C, the system again wakes up and begins a third actuation cycle, which as noted at 308, results in the successful delivery of a quantity of infusate. As further noted at 308, because the static friction was not broken at 302B, the third actuation cycle at 302C results in the plunger lurching forward 6 μm within the cartridge, thereby dispensing the quantity of infusate intended to be delivered at both 302B and 302C.

With reference to FIG. 5B, the infusion cycle 400 with stiction break detection begins at 402A, with a drive mechanism actuation cycle in which static friction is broken, such that a quantity of infusate is successfully delivered. At 404A, the system enters a low power consumption sleep duration for a preestablished length of time (e.g., 4.5 mins). Thereafter, unlike the conventional infusion cycle 300, at 402B, the infusion cycle 400 with stiction break detection monitors a force between the drive mechanism and the plunger for a decrease in a rate of the force over time during the drive mechanism actuation cycles, thereby positively indicating a threshold of motion between the plunger and medicament cartridge. Accordingly, the infusion cycle 400 of the present disclosure is “smart” in that it is able to confirm infusate delivery. This is confirmed at 406, in which it is noted that a quantity of infusate was successfully delivered.

At 404B, the low-power sleep duration 404 is calculated based on advancement of the actuator following the stiction break (e.g., as measured by the sensor 130). In an embodiment, the sleep duration can be correlated to the distance traveled by the drive mechanism, e.g., sleep for 4.5 minutes after advancement of the actuator 4.5 μm. Following the sleep duration 404B, a subsequent actuation cycle 402C is initiated at least until a threshold of motion of the plunger is established and infusate delivery is confirmed.

In the graphical representations (depicted in FIGS. 6A-B), time (t) is depicted along the x-axis, while a quantity of delivered infusate (Q) is depicted along the y-axis. Actuation of respective drive mechanisms (and low power consumption sleep durations therebetween) are also depicted along the x-axis. With reference to FIG. 6A, a total of four drive mechanism actuation cycles 302A, 302B, 302C, and 302D are depicted over the course of the conventional infusion cycle 300, with low power consumption sleep durations 304A, 304B, 304C therebetween.

As depicted, infusate is delivered on the first actuation cycle 302A, but the second actuation cycle 302B fails to deliver infusate, as the force produced by the drive mechanism fails to overcome the stiction between the plunger and the cartridge. Rather, static friction between the plunger and the cartridge is not overcome until the third actuation cycle 302C, where the plunger suddenly lurches forward to deliver a single large bolus of infusate. Again in the fourth actuation cycle 302C, a threshold of motion in the plunger is not established, as the stiction is not overcome.

In comparing FIGS. 6A and 6B, it can be seen that although the overall quantity of delivered infusate (Qₜₒₜₐₗ) is the same between the conventional infusion cycle 300 and the infusion cycle 400 with stiction break detection, the infusion cycle 400 of the present disclosure provides a more consistent flow of infusate over the same fixed duration of time. Moreover, the infusion cycle 400 of the present disclosure utilizes fewer drive mechanism actuation cycles and/or operates the drive mechanism for a shorter period of time, thereby representing a savings in power consumption over the comparative conventional infusion cycle 300.

Various embodiments of systems, devices, and methods have been described herein. These embodiments are given only by way of example and are not intended to limit the scope of the claimed subject matter. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed subject matter.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1. An infusion pump configured to identify a threshold of motion of a plunger within an infusate cartridge during delivery of an infusate, the infusion pump comprising:
a drive mechanism configured to actuate the plunger;
a force sensor configured to monitor a force between the drive mechanism and the plunger; and
a control unit configured to monitor data received from the force sensor to determine a decrease in a rate of the monitored force over time during actuation of the drive mechanism, thereby indicating a break in static friction between the plunger and the infusate cartridge, and to determine a low power consumption sleep duration based on an advancement of the actuator following the break in static friction.

2. The infusion pump of claim 1, wherein the control unit is further configured to initiate a low power consumption mode for the determined sleep duration.

3. The infusion pump of claim 1, wherein the control unit is further configured to initiate actuation of the drive mechanism following the determined sleep duration.

4. The infusion pump of claim 1, further comprising a battery.

5. The infusion pump of claim 4, wherein a length of the sleep duration is determined to reduce the number of actuation cycles of the drive mechanism within a fixed period of time to promote a more efficient use of the battery.

6. The infusion pump of claim 1, wherein the control unit is further configured to apply a low-pass filter to data representing the monitored force to reduce noise within the data.

7. The infusion pump of claim 1, wherein the control unit is further configured to calculate a derivative of data representing the monitored force to determine a rate in the force over time.

8. The infusion pump of claim 7, wherein the control unit is further configured to determine if the derivative of the data is less than a predefined threshold, thereby indicating a decrease in the rate of the force over time.

9. The infusion pump of claim 8, wherein the predefined threshold is between about −0.025 and about −2.0.

10. The infusion pump of claim 1, wherein the control unit is further configured to utilize a magnitude of the monitored force at the break in static friction to reduce noise during occlusion detection.

11. A method of identifying a threshold of motion of a plunger within an infusate cartridge during infusion of an infusate, the method comprising:
monitoring a force between a drive mechanism and a plunger for a decrease in a rate of the force over time during a steady-state actuation of the drive mechanism, thereby indicating a break in static friction between the plunger and the infusate cartridge; and
determining a low power consumption sleep duration based on an advancement of the actuator following the break in static friction.

12. The method of claim 11, further comprising initiating a low power consumption mode for the determined sleep duration.

13. The method of claim 11, further comprising initiating a steady-state actuation of the drive mechanism following the determined sleep duration.

14. The method of claim 11, wherein a length of the sleep duration is determined to reduce the number of steady-state actuation cycles of the drive mechanism within a fixed period of time to promote a more efficient use of electrical power.

15. The method of claim 11, further comprising applying a low-pass filter to data representing the monitored force to reduce noise within the data.

16. The method of claim 11, further calculating a derivative of data representing the monitored force to determine a rate in the force over time.

17. The method of claim 16, further determining if the derivative of the data is less than a predefined threshold, thereby indicating a decrease in the rate of the force over time.

18. The method of claim 17, wherein the predefined threshold is between about −0.025 and about −2.0.

19. The method of claim 11, further comprising utilizing a magnitude of the monitored force at the break in static friction to reduce noise during occlusion detection.

20. An infusion system, comprising:
an infusate cartridge including a plunger, the infusate cartridge configured to retain an infusate therein; and
an infusion pump configured to selectively receive the infusate cartridge and further configured to identify a break in static friction between the plunger and the infusate cartridge during an infusion of the infusate for the purpose of providing a more consistent flow of the infusate and promoting a more efficient use of energy, the infusion pump comprising:
a drive mechanism configured to actuate the plunger;
a force sensor configured to monitor a force between the drive mechanism and the plunger; and
a control unit configured to—
apply a low-pass filter to data received from the force sensor to reduce noise within the data,
calculate a derivative of data to determine a rate in the force over time,
determine if the derivative of the data is less than a predefined threshold, thereby indicating a break in static friction between the plunger and the infusate cartridge, and
determine a low power consumption sleep duration based on an advancement of the actuator following the break in static friction.
